(19) United States
(12) Patent Application Publication
(10) Pub. No.: US 2025/0153002 A1
(43) Pub. Date: May 15, 2025

(54) SYSTEM AND METHOD TO PREDICT PERFORMANCE ON STRUCTURED PHYSICAL ACTIVITY USING WEARABLE SENSORS

(71) Applicant: Georgia Tech Research Corporation, Atlanta, GA (US)

(72) Inventors: Aprameya Satish, Atlanta, GA (US); David Lin, Atlanta, GA (US); Omer Inan, Atlanta, GA (US); Alessio Medda, Marietta, GA (US); Kristine Richardson, Atlanta, GA (US)

(21) Appl. No.: 18/945,291

(22) Filed: Nov. 12, 2024

Related U.S. Application Data
(60) Provisional application No. 63/597,929, filed on Nov. 10, 2023.

(51) Int. Cl. A63B 24/00

(52) U.S. Cl. CPC A63B 24/0062; A63B 2220/12

(57) ABSTRACT

An exemplary system and method employing physiological sensor fusion and trained artificial intelligence models to predict/estimate a time-to-completion for a user undergoing a structured activity. The time-to-completion may be determined at a given segment of the activity as defined only by the physiological sensor measurement. The exemplary system and method may provide individualized fitness information for a customized military training regimen or athlete programs, to provide comprehensive and localized snapshots of physical performance based on the entire history of the event/activity.

### RELATED APPLICATION

This U.S. application claims priority to, and the benefit of, U.S. Provisional Patent Application No. 63/597,929, filed Nov. 10, 2023, entitled “SYSTEM AND METHOD TO PREDICT PERFORMANCE ON STRUCTURED PHYSICAL ACTIVITY USING WEARABLE SENSORS,” which is incorporated by reference herein in its entirety.

### STATEMENT REGARDING FEDERALLY SPONSORED RESEARCH OR DEVELOPMENT

This invention was made with government support under N00014-20-1-2137, awarded by the Office of Naval Research. The government has certain rights in the invention.

### BACKGROUND

Performance metrics are important in structured physical activities and are a driving factor in adjusting training regimens (e.g., running, cycling, marching). Among athletes and military personnel, one important performance indicator is the time taken to complete a structured activity. Obstacles courses, marathon courses, and cross-country courses are complex environments and may include surfaces of grass, trails, and roadways, passing through woodlands, open country, and urban centers.

There is a benefit to having improved estimates of training regimens.

### SUMMARY

An exemplary system and method are disclosed employing physiological sensor fusion (e.g., heart rate signal, temperature signal, accelerometer signal, body signal, or a combination thereof) and trained artificial intelligence (AI) models to predict/estimate a time-to-completion (TTC) for a user (e.g., athletes, military personnel) undergoing a structured activity. As used herein, a structured activity may be a ruck march, runs over a course length, or cycling. The exemplary system and method may provide individualized fitness information for a customized military training regimen (e.g., ruck marches, runs) or athlete programs (cross-country, track), to provide comprehensive and localized snapshots of physical performance based on the entire history of the event/activity.

Examples of multi-modal physiological sensor fusion include, but are not limited to electrocardiogram sensors, electromyogram sensors, blood pressure sensors, motion sensors, accelerometer sensors, etc. In some embodiments, the exemplary system and method may be incorporated into current state-of-the-art fitness trackers, or may be used as a standalone fitness tracker. In some embodiments, the exemplary system and method may not rely on a global positioning system (GPS)-using only physiological signals and localized data—for the performance prediction. Physiological sensors may have a lower associated manufacturing cost as compared to network devices. Physiological sensors can also operate in environments lacking in global-positioning signals (GPS), e.g., indoors, or mobile-positioning signals (MPS) signals, e.g., woodland areas.

Current state-of-the-art fitness trackers that employ only one sensor type may not be able to predict performance at a similar level of accuracy to facilitate accurate TTC measurements due to the lack of sensor fusion.

In an aspect, method (for an analysis system) is disclosed comprising receiving, by a processor, signals from a wearable sensor device worn by a user during an activity, wherein the activity is defined by the user moving through a plurality of geographic checkpoints at a first location, including a first checkpoint and a second checkpoint; and determining, via one or more trained machine learning (ML) models or a model derived therefrom, (e.g., at each of the plurality of checkpoints including the first checkpoint and the second checkpoint), an estimated time-to-completion (TTC) determined at a given segment of the activity (e.g., time duration) as the user in moving a distance from a start position to a position in the given segment through the plurality of geographic checkpoints as defined by the segments, wherein each respective trained AI model of the one or more trained ML models was trained on one or more physiological signals for a set of users (preferably a different set of users than the current user) moving through a set of geographic checkpoints up and including to the segment, wherein the estimated time-to-completion of the activity (e.g., a duration value) or an estimated complete time derived therefrom is outputted to be displayed at the wearable sensor device or an external remote device.

The “time to completion” refers to the time taken for an individual to complete the entire event, or a pre-defined portion of the event as defined by the trained AI model. The TTC is preferably determined as the estimate either of how much time is remaining for a person to complete the full event, i.e., total TTC is the elapsed time, or estimate time taken to complete the full event, from event start. In alternative embodiments, TTC may predict TTC on a per-segment basis.

In some embodiments, the estimated time-to-completion of the activity (e.g., a duration value) or the estimated complete time derived therefrom is outputted to a cloud network, wherein the cloud network transmits the estimated time-to-completion of the activity or the estimated complete time to the wearable sensor device or the remote device for display.

In some embodiments, the determining the estimated time-to-completion (TTC) determined for the given segment of the activity (e.g., time duration) is performed at the wearable sensor device.

In some embodiments, the physiological signals are selected from the group consisting of a heart rate signal, a temperature signal, an accelerometer signal (e.g., triaxial accelerometry), a body signal (e.g., for gait-associated measure), or a combination thereof (e.g., wherein the one or more trained ML models are further trained using an estimated parameter (e.g., estimated core temperature)).

In some embodiments, the trained AI models include a machine learning model or a neural network model (e.g., wherein the determining of the estimated time-to-completion (TTC) further employs a physics-based model, e.g., a cadence-based model, a stride-length-based model, and a mean TTC-based model).

In some embodiments, the estimated time-to-completion of the activity is defined as an average predicted duration of remaining time to complete the activity as of that given segment, wherein the checkpoint or segment is determined only by the signals from the wearable sensor device. In some embodiments, the determining the estimated time-to-completion (TTC) for the given segment of the activity (e.g., time duration) is performed at a computing device located in cloud infrastructure.

In some embodiments, the determining the estimated time-to-completion (TTC) of the given segment of the activity (e.g., time duration) is performed at a remote computing device.

In an aspect, an analysis system is disclosed comprising a processor; and a memory having instructions stored thereon, wherein execution of the instructions by the processor causes the processor to receive, by a processor, signals from a wearable sensor device worn by a user during an activity, wherein the activity is defined by the user moving through a plurality of geographic checkpoints at a first location, including a first checkpoint and a second checkpoint; and determine, via one or more trained AI models or a model derived therefrom, (e.g., at each of the plurality of checkpoints including the first checkpoint and the second checkpoint), an estimated time-to-completion (TTC) determined at a given segment of the activity (e.g., time duration) as the user in moving a distance from a start position to a position in the given segment through the plurality of geographic checkpoints as defined by the segments, wherein each respective trained AI model of the one or more trained ML models was trained on one or more physiological signals for a set of users (preferably a different set of users than the current user) moving through a set of geographic checkpoints up and including to the segment, wherein the estimated time-to-completion of the activity (e.g., a duration value) or an estimated complete time derived therefrom is outputted to be displayed at the wearable sensor device or an external remote device.

In some embodiments, the estimated time-to-completion of the activity (e.g., a duration value) or an estimated complete time derived therefrom is outputted to be displayed on a network interface.

In some embodiments, the wearable sensor device includes one or more sensors configured to measure the physiological signals for a set of users moving through a set of geographic checkpoints at a plurality of locations.

In some embodiments, the physiological signals are selected from the group consisting of a heart rate signal, a temperature signal, an accelerometer signal (e.g., triaxial accelerometry), a body signal (e.g., for gait-associated measure), or a combination thereof (e.g., wherein the one or more trained ML models are further trained using an estimated parameter (e.g., estimated core temperature)).

In some embodiments, the determining the estimated time-to-completion (TTC) of the given segment of the activity (e.g., time duration) is performed at a computing device located in cloud infrastructure.

In some embodiments, the determining the estimated time-to-completion (TTC) of the given segment of the activity (e.g., time duration) is performed at a remote computing device.

In an aspect, a non-transitory computer-readable medium is disclosed having instructions stored thereon, wherein execution of the instructions by a processor causes the processor to receive, by a processor, signals from a wearable sensor device worn by a user during an activity, wherein the activity is defined by the user moving through a plurality of geographic checkpoints at a first location, including a first checkpoint and a second checkpoint; and determine, via one or more trained machine learning (ML) models or a model derived therefrom, (e.g., at each of the plurality of checkpoints including the first checkpoint and the second checkpoint), an estimated time-to-completion (TTC) determined at a given segment of the activity (e.g., time duration) as the user in moving a distance from a start position to a position in the given segment through the plurality of geographic checkpoints as defined by the segments, wherein each respective trained AI model of the one or more trained ML models was trained on one or more physiological signals for a set of users (preferably a different set of users than the current user) moving through a set of geographic checkpoints up and including to the segment, wherein the estimated time-to-completion of the activity (e.g., a duration value) or an estimated complete time derived therefrom is outputted to be displayed at the wearable sensor device or an external remote device.

In some embodiments, the estimated time-to-completion of the activity (e.g., a duration value) or an estimated complete time derived therefrom is outputted to be displayed on a network interface.

In some embodiments, the wearable sensor device includes one or more sensors configured to measure the physiological signals for a set of users moving through a set of geographic checkpoints at a plurality of locations.

In some embodiments, the physiological signals are selected from the group consisting of a heart rate signal, a temperature signal, an accelerometer signal (e.g., triaxial accelerometry), a body signal (e.g., for gait-associated measure), or a combination thereof (e.g., wherein the one or more trained ML models are further trained using an estimated parameter (e.g., estimated core temperature)).

In some embodiments, the determining the estimated time-to-completion (TTC) of the given segment of the activity (e.g., time duration) is performed at a computing device located in cloud infrastructure.

In some embodiments, the determining the estimated time-to-completion (TTC) of the given segment of the activity (e.g., time duration) is performed at a remote computing device.

### BRIEF DESCRIPTION OF DRAWINGS

FIGS. 1A-1C each shows an example analysis system configured with trained machine learning (ML) models and wearable sensor devices in accordance with an illustrative embodiment. FIG. 1B employs an additional cloud network. FIG. 1C employs an additional computing device located on cloud infrastructure.

FIG. 2 shows an example operation flow of the exemplary method in accordance with an illustrative embodiment.

FIGS. 3A-3B each shows an example executable model for time-to-completion (TTC) estimation of an activity. FIG. 3A shows an example executable model for time-to-completion (TTC) estimation of a ruck march. FIG. 3B shows the same executable model for TTC estimation of cycling activity.

FIGS. 4A-4H show the experimental and evaluation results of the exemplary method (i.e., model) with the state-of-the-art (SoA) TTC estimation models. FIG. 4A shows the exemplary model for TTC estimation implemented in the experiments. FIG. 4B shows the feature extraction pipeline of the exemplary model in the experiments. FIG. 4C shows the root mean square error (RMSE) and maximum absolute error (MAE) for the TTC estimations of the exemplary method and SoA models. FIG. 4D shows the modified Bland-Altman and correlation plots for estimated TTC using two exemplary methods, e.g., random forest (RF) model with acceleration (ACC) feature only and RF model with all features. FIG. 4E shows the TTC RMSE while varying the input feature matrix or the number of models trained to predict TTC. FIG. 4F shows the top 15 features across all trained models. FIG. 4G shows the relationship between high vertical acceleration standard deviation and TTC, color-coded by the percentage of windows that the participants (i.e., subjects) spent running. FIG. 4H shows the percentage of participants at each checkpoint whose absolute TTC prediction error exceeded a threshold of 10 or 15 minutes.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. All references cited and discussed in this specification are incorporated herein by reference in their entirety and to the same extent as if each reference was individually incorporated by reference.

### Example Systems

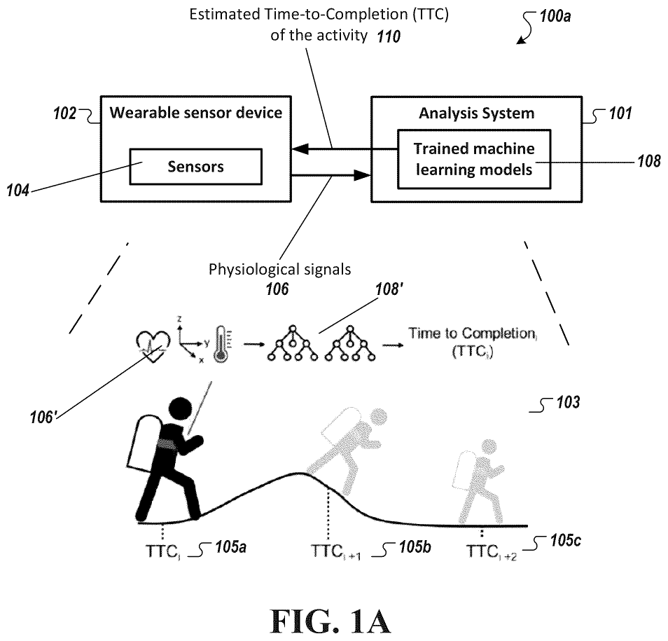

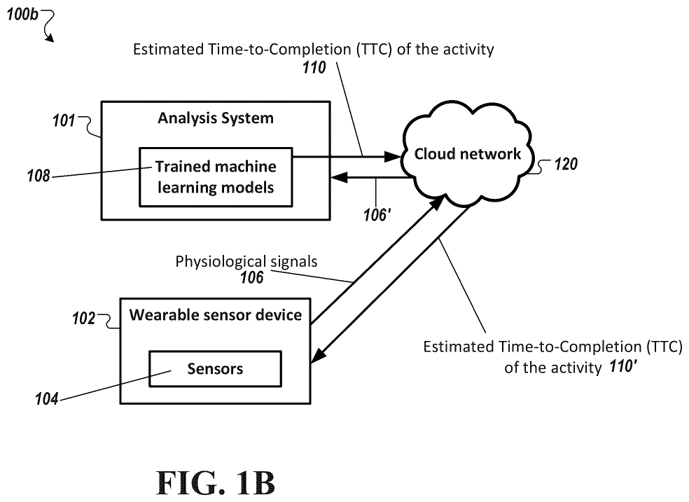

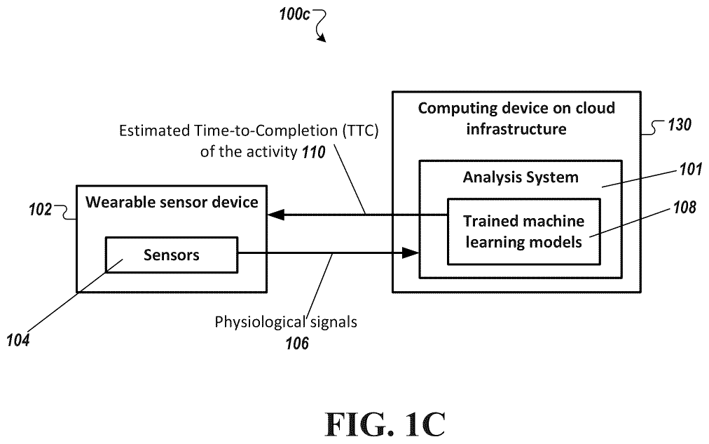

FIGS. 1A-1C each shows an example system 100 (shown as 100a, 100b, 100c) configured with an analysis system 101 and a wearable sensor device 102 in accordance with an illustrative embodiment. FIG. 1B employs an additional cloud network 120. FIG. 1C employs an additional computing device located on cloud infrastructure 130.

Example System #1. In the example shown in FIG. 1A, the example system 100a is configured with an analysis system 101 and a wearable sensor device 102. The wearable sensor device 102 is worn by a user during activity 103 (e.g., walking, sprinting) to generate physiological signals 106 (shown as 106′). The activity 103 may be defined by the user moving through a plurality of geographic checkpoints at a first location, including a first checkpoint and a second checkpoint, e.g., through an obstacles course, marathon course, and cross-country courses are complex environments and may include surfaces of grass, trails, and roadways, passing through woodlands, open country, and urban centers.

In FIGS. 1A-1C, the wearable sensor device 102 may include a plurality of sensors 104 configured to measure the physiological signals 106 while the user is performing activity 103. Examples of sensor 104s include, but are not limited to, electrocardiogram sensor, electromyogram sensor, blood pressure sensor, motion sensor, accelerometer sensor, etc. The sensors can acquire physiological signals 106 selected from the group consisting of a heart rate signal, a temperature signal, an accelerometer signal (e.g., triaxial accelerometry), a body signal (e.g., for gait-associated measure), or a combination thereof.

In FIG. 1A, the analysis system 101, operatively coupled with the wearable sensor device 102 within the device, may receive the physiologic signals 106 from the wearable sensor device 102. Trained AI models 108 (shown as 108′), operating on the analysis system 101, may determine an estimated time-to-completion (TTC) determined at a given segment of the activity (e.g., time duration 105a-105c) in moving a distance from a start position to a position in a segment through the plurality of geographic checkpoints as defined by the segment using the physiological signals 106 as input to the trained AI model 108. To this end, the prediction at the first segment may estimate the TTC-remaining time-between a position in the first segment and a final position after the last segment using signals acquired up to the position in the first segment. And, the prediction at the second segment may estimate the TTC—remaining time—between a position in the second segment and the final position in the last segment using signals acquired up the position in the second segment and not yet including the measurements in the third segment located after the second segment. And the prediction at the n segment (where there are n segment total) may estimate the TTC—remaining time—at a position in n segment.

The trained AI models 108 may output an estimated time-to-completion 110 of the activity 103 (e.g., a duration value) or an estimated complete time derived therefrom to the wearable sensor device 102 for display. In some embodiments, multiple AI models 108 may be employed to provide an estimated time-to-completion 110 at a segment of the overall activity, e.g., between pre-defined checkpoints in the activity. Only one of the plurality of estimated time-to-completion 110 may be provided as a total estimate for the time to complete the activity depending on the sensor measurement corresponding to the segment.

To determine the estimated time-to-completion, the trained AI models 108 may include an ML model or a neural network model. The trained AI models 108 may further employ a physics-based model, e.g., a cadence-based model, a stride-length-based model, and a mean TTC-based model. Multiple AI models 108 may be employed to provide an estimated time-to-completion 110 as defined by the segment of the overall activity. For example, each random forest or other AI model may use physiological information collected up to that checkpoint to predict TTC (how long someone will take to complete the full march). Hence, over the full march, there would be multiple random forest or AI models used to do the prediction. The estimated time-to-completion 110 of the activity 103 may be defined as an average duration of remaining time of the estimated time-to-completion (shown as 105a-105c) of the activity at each of the plurality of checkpoints. In some embodiments, the estimated TTC (shown as 105a-105c) is a predicted remaining duration to complete the activity from the current position to the finish position using physiological signals acquired up to the checkpoint. In the system 100a, the operation where the trained AI models 108 determines the respective estimated time-to-completion (TTC) at the segment of the activity in moving the distance from the start position to the current position in the segment through the plurality of geographic checkpoints as defined the segment may be performed at the wearable sensor device 102.

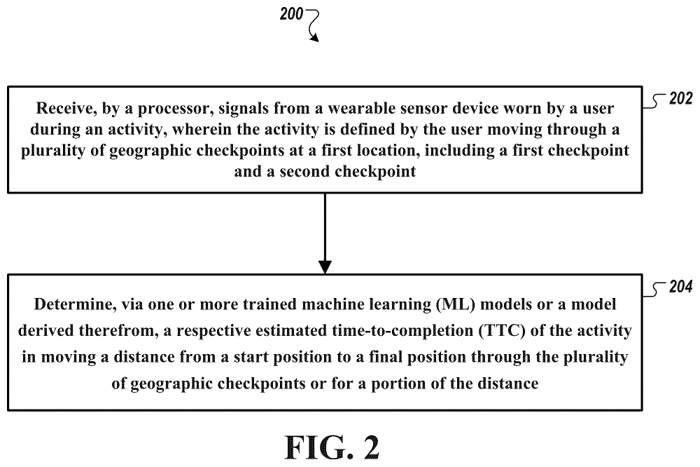

Example System #2. In the example shown in FIG. 1B, the system 100b is configured with an analysis system 101, a wearable sensor device 102, and a cloud network 120. In FIG. 2B, the wearable sensor device 102, worn by a user during an activity (e.g., walking, sprinting), is configured to generate physiological signals 106 and transmit them to the cloud network 120. The activity may be defined by the user moving through a plurality of geographic checkpoints at a first location, including a first checkpoint and a second checkpoint. The wearable sensor device 102 may comprise sensors 104 configured to measure the physiological signals 106 while the user is performing the activity.

The analysis system 101, via the cloud network 120, may receive the physiologic signals 106. The trained AI models 108, operating on the analysis system 101 and using the physiological signals 106, may determine an estimated time-to-completion (TTC) at a given segment of the activity in moving a distance from a start position to a current position in the given segment through the plurality of geographic checkpoints as defined by the segment. The trained AI models 108 may then output an estimated time-to-completion 110 (shown as 110′) of the activity (e.g., a duration value) or an estimated complete time derived therefrom to the cloud network 120, wherein the cloud network 120 may transmit the estimated time-to-completion 110′ of the activity to the wearable sensor device 102 for display. In the system, 100b, the operation where the trained AI models 108 determines the estimated time-to-completion (TTC) at the given segment of the activity may be performed at a remote computing device via the cloud network 120.

Example System #3. In the example shown in FIG. 1C, the system 100c is configured with a wearable sensor device 102 and an analysis system 101 operating on a computing device on cloud infrastructure 130. The wearable sensor device 102, worn by a user during an activity (e.g., walking, sprinting), may generate physiological signals 106. The activity may be defined by the user moving through a plurality of geographic checkpoints at a first location, including a first checkpoint and a second checkpoint.

The wearable sensor device 102 may comprise sensors 104 configured to measure the physiological signals 106 while the user is performing the activity. The analysis system 101, operating on the computing device 130, may receive the physiologic signals 106. The trained AI models 108, operating on the analysis system 101 and using the physiological signals 106, may determine an estimated time-to-completion (TTC) at a given segment of the activity in moving a distance from a start position to a current position in the given segment through the plurality of geographic checkpoints as defined by the segment.

The trained AI models 108 may then output an estimated time-to-completion 110 of the activity (e.g., a duration value) or an estimated complete time derived therefrom to the wearable sensor device 102 for display. In the system 100c, the operation where the trained AI models 108 determines the respective estimated time-to-completion (TTC) of each respective segment of the activity in moving the distance from the start position to the final position in the segment through the plurality of geographic checkpoints defining the segment may be performed at the computing device located in cloud infrastructure 130.

### Example Method

FIG. 2 shows an example operation flow 200 for the exemplary method, which may include 2 steps.

At step 202, the exemplary method may receive, by a processor, signals from a wearable sensor device worn by a user during an activity, wherein the activity is defined by the user moving through a plurality of geographic checkpoints at a first location, including a first checkpoint and a second checkpoint.

At step 204, the exemplary method may determine, via one or more trained machine learning (ML) models or a model derived therefrom, (e.g., at each of the plurality of checkpoints, including the first checkpoint and the second checkpoint), an estimated time-to-completion (TTC) determined at a given segment of the activity (e.g., time duration) as the user in moving a distance from a start position to a position in the given segment through the plurality of geographic checkpoints as defined by the segments, wherein each respective trained AI model of the one or more trained ML models was trained on one or more physiological signals for a set of users (preferably a different set of users than the current user) moving through a set of geographic checkpoints up and including to the segment.

In some embodiments, the estimated time-to-completion of the activity or the estimated complete time derived therefrom is outputted to a cloud network, wherein the cloud network transmits the estimated time-to-completion of the activity or the estimated complete time to the wearable sensor device or the remote device for display.

In some embodiments, the step of determining the estimated time-to-completion (TTC) at the segment of the activity is performed at the wearable sensor device.

In some embodiments, the physiological signals are selected from the group consisting of a heart rate signal, a temperature signal, an accelerometer signal, a body signal.

In some embodiments, the trained ML models include a machine learning model or a neural network model.

In some embodiments, the step of determining the estimated time-to-completion (TTC) at the given segment of the activity is performed at a computing device located in cloud infrastructure.

In some embodiments, the step of determining the estimated time-to-completion (TTC) at the given segment of the activity in moving the distance from the start position to the current position in the segment through the plurality of geographic checkpoints as defined by the segment is performed at a remote computing device.

### Example TTC Estimation Model

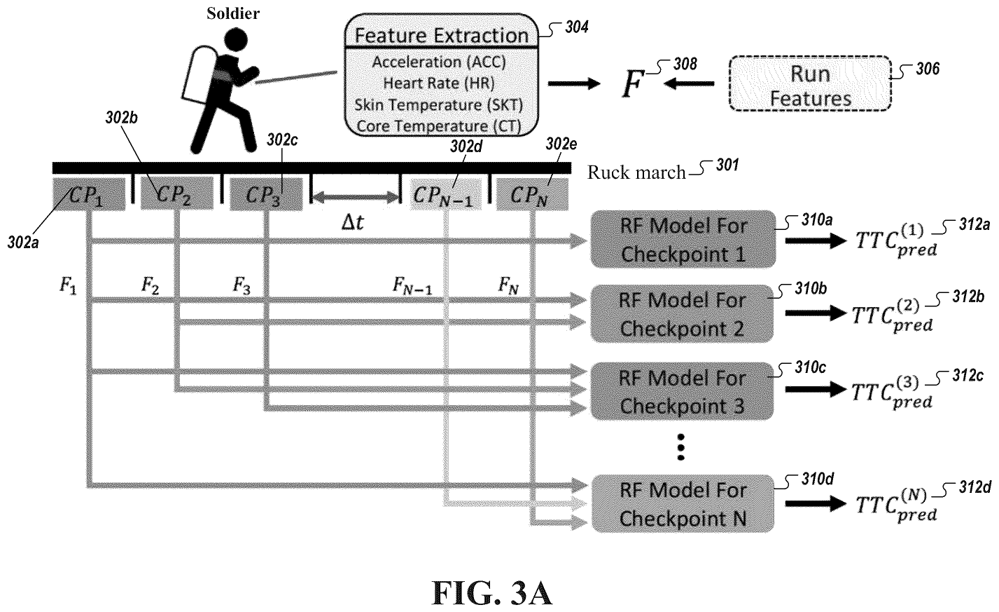

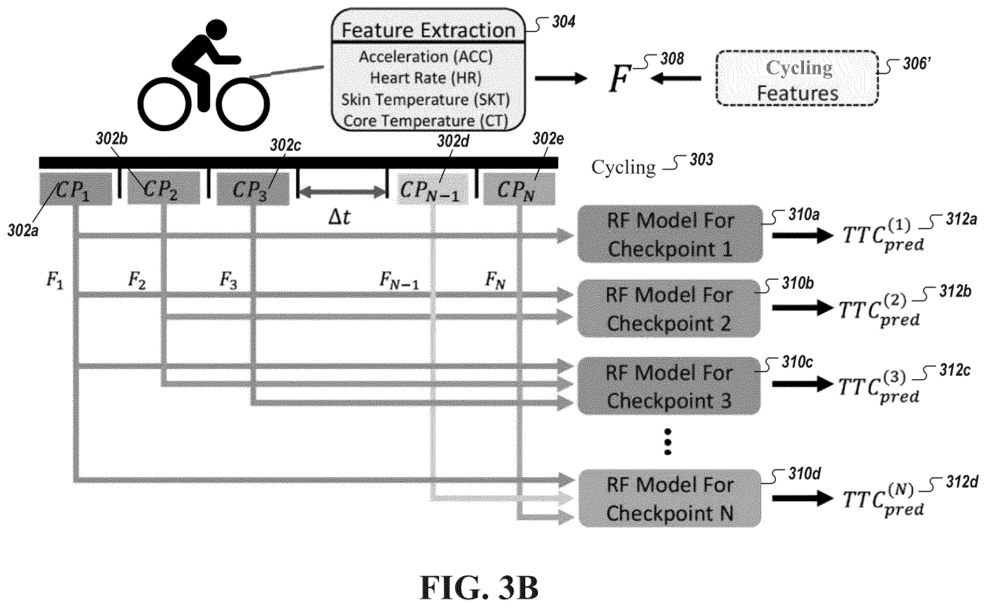

FIGS. 3A and 3B each show the executable model for TTC estimation. In FIG. 3A, the model defines a march or run (e.g., 12-mile ruck march) 301 for each subject (e.g., athlete, trainee, soldier). In FIG. 3B, the activity is performed on a bicycle.

In each of FIGS. 3A and 3B, the model is divided into equal segments of length At (e.g., 302a-302e), called checkpoints CP (shown as CP₁, CP₂, . . . , CP_{N}). Signals within each checkpoint CPᵢ are then used to calculate features 304 and subsequently combined with run features 306 to form the feature vector 308 (denoted as Fᵢ or F₁, F₂, . . . , F_{N}) for CPᵢ. The associated model at CPᵢ (e.g., 310a-310d) is then trained on the feature set [Fᵢ, Fᵢ₋₁, F₁] corresponding to features from the current, previous, and baseline checkpoints to estimate the TTC at that point in time (e.g., 312a-312d).

The march or run duration 301 is first divided into standard epochs. The epochs are referred to as “checkpoints” (CP), which serve as reference gates for predicting TTC. For each subject, features Fᵢ (shown as 308) for each checkpoint CPᵢ is formed using both the signals 304 measured at that checkpoint and from the run features 306. These “global” run features 306 may provide previous knowledge of soldier fitness.

In the exemplary model, an epoch (e.g., at every 10 minutes) is chosen as the checkpoint length to provide TTC estimates at a pre-defined portion of the total expected duration (e.g., approximately every 5% of the total expected march duration). A parametric study of checkpoint length was conducted and later described herein, and a 10-minute checkpoint length may offer a reasonable compromise between computational cost and prediction accuracy, with shorter checkpoints performing similarly and longer checkpoints reducing prediction accuracy.

The total time to completion (TTC) 312a-312d may then be estimated at each checkpoint by inputting features 308 from multiple checkpoints into a distinct random forest regression model [19]. Specifically, the features from the current checkpoint (Fᵢ), previous checkpoint (Fᵢ₋₁), and baseline checkpoint (F₁) may be concatenated together and used as input to the corresponding model. The baseline feature matrix may be set as the feature matrix before the first checkpoint.

In an example, random forest regression may be chosen for its ability to explain non-linear relationships in an explainable model using feature importance [20]. For the exemplary model, the Gini importance index may be used to estimate the relative importance of each feature to TTC estimation performance [19].

Several engineered features may be computed, e.g., using the raw accelerometer (e.g., from an IMU having multiple axes of acceleration) and heart rate data that were acquired from the sensors. These features may include statistical indicators such as means, standard deviations, signal power, etc., e.g., for a standard deviation of vertical acceleration, a total power of vertical acceleration, mean step time, cadence, step count, power of vertical acceleration between the 0-3 Hz frequency band, the 5-mile run time (contextual information about prior fitness), heart rate slope, approximate entropy of the Antero-Posterior acceleration, vertical Detail (Level 1) Wavelet Coefficient, power of medio-lateral acceleration between the 8-20 Hz frequency band, standard deviation of the medio-lateral acceleration, SD1/SD2 ratio (heart rate parameter), kurtosis of the antero-posterior acceleration, standard deviation of step time, among others.

In some embodiments, the trained AI model may employ at least one of these features. In some embodiments, the trained AI model may employ at least two of these features.

The standard deviation of vertical acceleration may be determined using the standard deviation function of sensor measurement (vertical acceleration) from a start position or time to a current position or time.

The total power of vertical acceleration may be the power of sensor measurement (vertical acceleration) from a start position or time to a current position or time.

The mean and standard deviation step time may be determined by a mean function (e.g., average) and a standard derivation function of a duration determined between spikes in sensor measurement (e.g., one channel of the sensors) from a start position or time to a current position or time.

Cadence is a measurement of how many steps a person takes per minute and may be determined as an average or standard deviation in the number of steps per minute.

The step count may be determined as the count of spikes in the measurement from a start position or time to a current position or time.

The power of vertical acceleration between the 0-3 Hz frequency band may be determined by a power function defined at a 0-3 Hz band of an FFT of sensor measurement from a start position or time to a current position or time.

The heart rate slope may be determined as a moving-average slope defined by fiduciary points in the heart signal (e.g., peak heart rate) for a time window, e.g., 1 minute. In some embodiments, the heart rate slope is determined in successive windows over the window length to provide a measure of heart rate dynamics.

The approximate entropy of the Antero-Posterior acceleration may be determined as an entropy function (e.g., Lyapunov) of sensor measurement from a start position or time to a current position or time.

The vertical detail (Level 1) wavelet coefficient may be determined as a coefficient for wavelet function applied to the sensor measurement from a start position or time to a current position or time.

The power of medio-lateral acceleration between the 8-20 Hz frequency band may be determined using a power function at 0-3 Hz band of an FFT of sensor measurement from a start position or time to a current position or time.

The standard deviation of the medio-lateral acceleration may be determined using a standard deviation function at the 0-3 Hz band of an FFT of sensor measurement from a start position or time to a current position or time.

The SD1/SD2 ratio (heart rate parameter) may be determined as a ratio of a first slope determined from the peaks in the heart signal to a baseline slope determined in the heart signal excluding the peaks.

The kurtosis of the antero-posterior acceleration is a kurtosis value of sensor measurement from a start position or time to a current position or time.

The 5-mile run time (minutes) is a prior measurement value, e.g., collected prior to a ruck march across the training cohort, and represents the time taken for a subject to complete a 5-mile run over a flat track. The 5-mile run can represent contextual information on the subject's prior fitness that allows our models to converge better on personalized TTC estimates. The 5-mile run-time can be supplemented with other contextual information as well (e.g., number of reps of PT exercises, acclimatization to weather, perspiration rate, etc.).

Example Machine Learning Model Training. In addition to random forest, other machine learning and AI models may be used, including supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

It is to be understood that the methods and systems are not limited to specific synthetic methods, specific components, or to particular compositions. It is also to be understood that the terminology used herein is for the purpose of describing particular implementations only and is not intended to be limiting.

### Experimental Results and Additional Examples

A study was conducted to develop and evaluate a model that uses signals from a multi-modal wearable sensor to predict TTC for soldiers undergoing a 12-mile structured ruck march. Predictions were made at discrete time points (checkpoints) throughout the march using features from skin temperature, heart rate, estimated core temperature, and triaxial accelerometry.

To utilize the structured nature of these marches, separate models were trained at each checkpoint using features from both the current and past checkpoints. By 120 minutes (⅔ of the expected 180-minute completion time), the study achieved a TTC RMSE of 7.12 minutes and an MAE of 5.21 minutes using the model. Integral to TTC estimation accuracy were gait-related features such as the standard deviation of vertical acceleration. Features such as heart rate slope and performance metrics from prior exercises minimally improved accuracy. The deployment of this model may enable continuous monitoring of performance metrics for online TTC estimation.

The study used data collected under a protocol approved by the Medical Research and Development Command Institutional Review Board (Protocol number M-10720). These data were collected during 12-mile loaded rucksack marches and 5-mile runs during the Ranger Assessment and Selection Program (RASP) at Fort Benning, Columbus, Georgia. Soldiers were included in the analysis if they participated in both exercises and if their 12-mile march TTC was within an acceptable range. The study was comprised predominantly of male soldiers (23±4) years of age, with an average height (of 1.77±0.08) meters and a body weight (of 78.1±10.4) kg. Each subject participated in a single march event.

The study used data from 468 soldiers who underwent both a 12-mile march on a predetermined route and a 5-mile run on a paved track. Soldiers needed to complete the 12-mile march within a 180-minute timeframe and were free to use their favorite combination of military marching and running strategies. Soldiers were equipped with their army combat uniform and carried a loaded rucksack and a weapon totaling 14 kg in combined weight. For the 5-mile run, soldiers needed to complete the exercise in 40 minutes. Soldiers did not carry a load during this exercise.

During each exercise, participants were equipped with a custom torso-worn wearable sensor (Open Body Area Network Physiological Status Monitor, OBAN-PSM) strapped around the chest to measure and log heart rate (HR), skin temperature (SKT), and acceleration (ACC) signals [18]. HR was captured using a custom dry electrode electrocardiogram (ECG) sensor around the chest.

Although the ECG was sampled at a much higher rate (512 Hz), the sensor did not store the raw ECG waveform and instead outputs a time-averaged HR estimate every 5 seconds (0.2 Hz). Tri-axial acceleration was captured using an accelerometer (ADXL362 chip, ±8 g; Analogue Devices, Norwood, Massachusetts, USA) and sampled at 128 Hz. Skin temperature was sampled at 0.5 Hz. Core temperature (CT) was subsequently derived using a Kalman-filter architecture applied to heart rate measurements known as the ECTemp algorithm developed by Buller et al. [14].

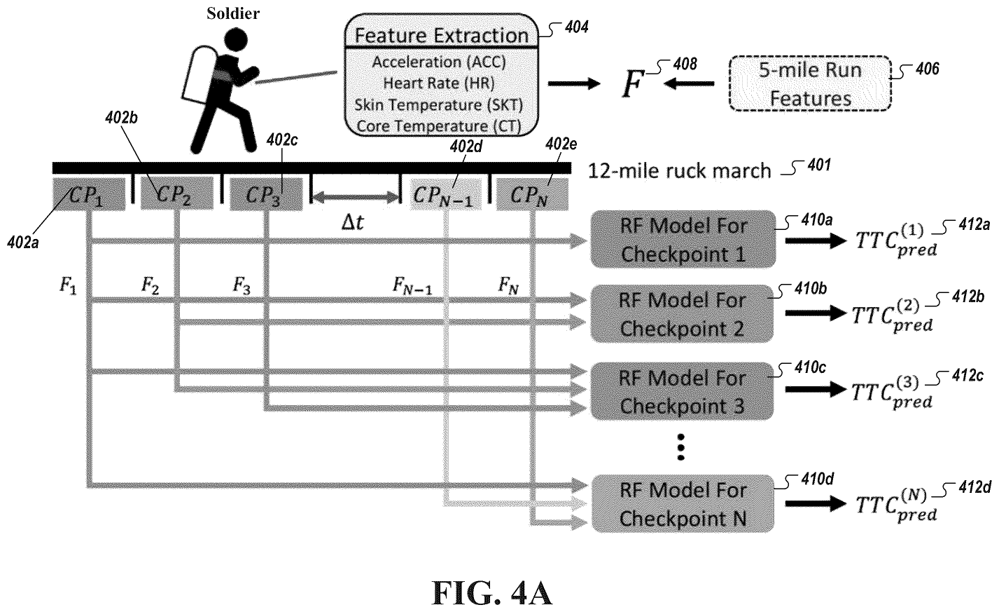

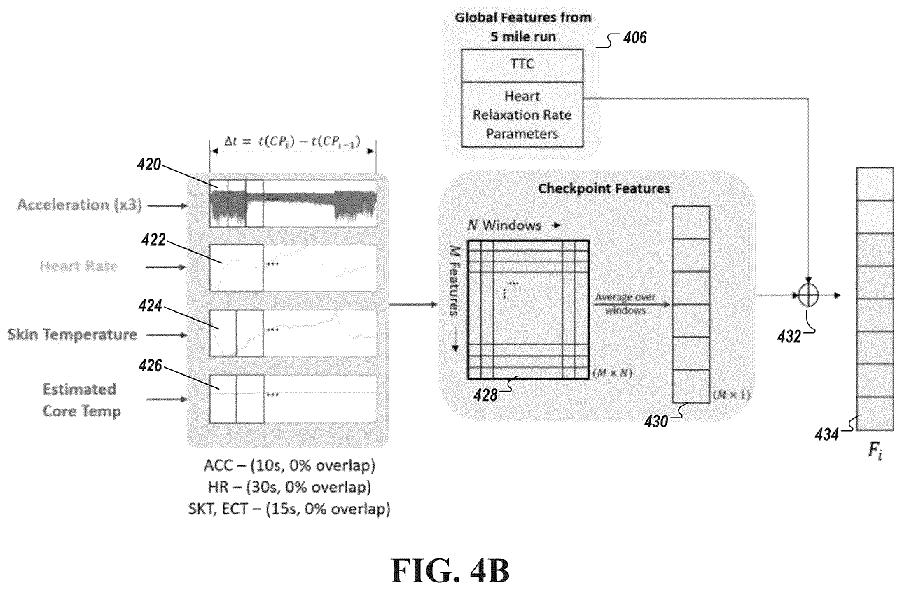

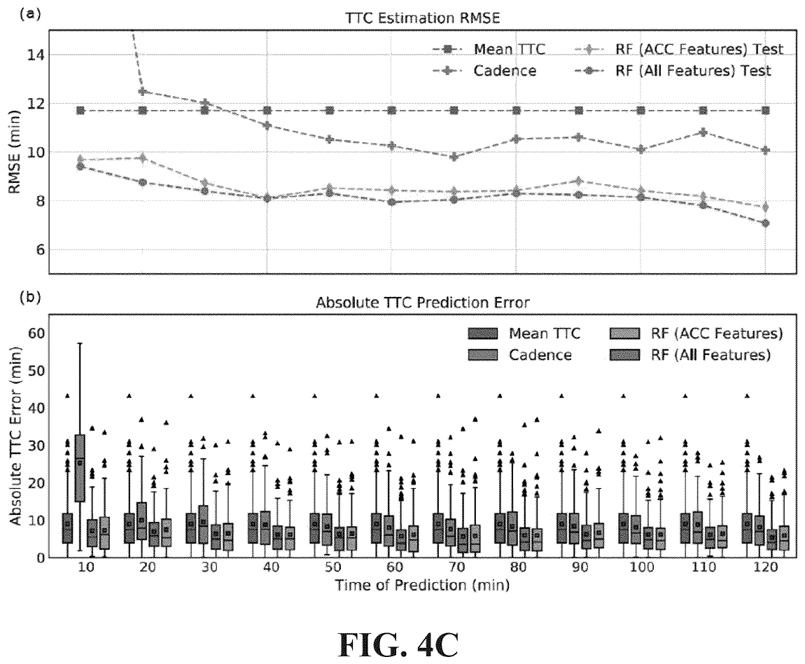

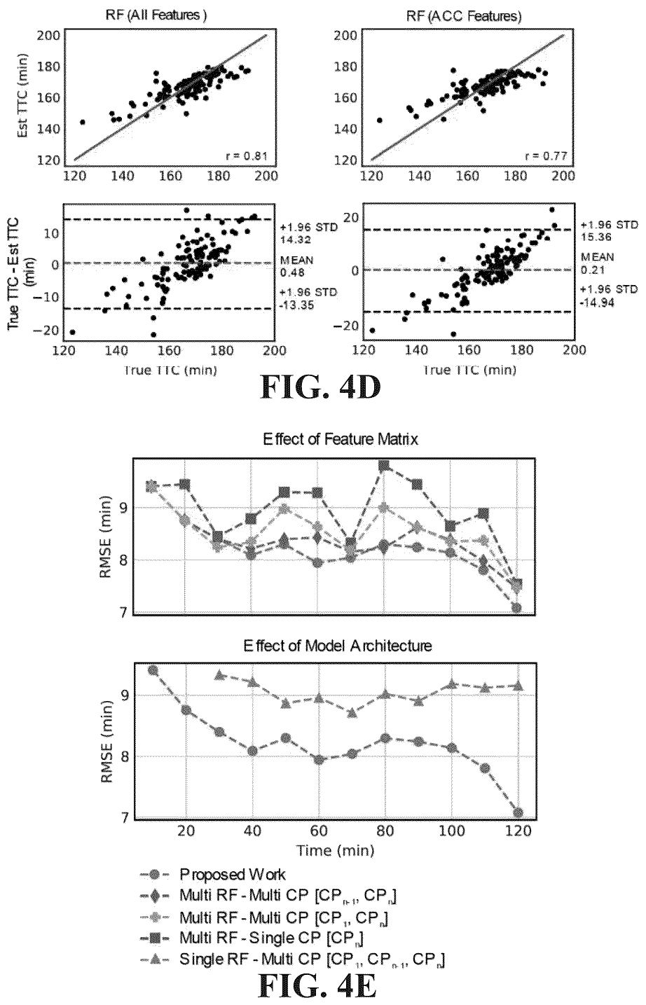

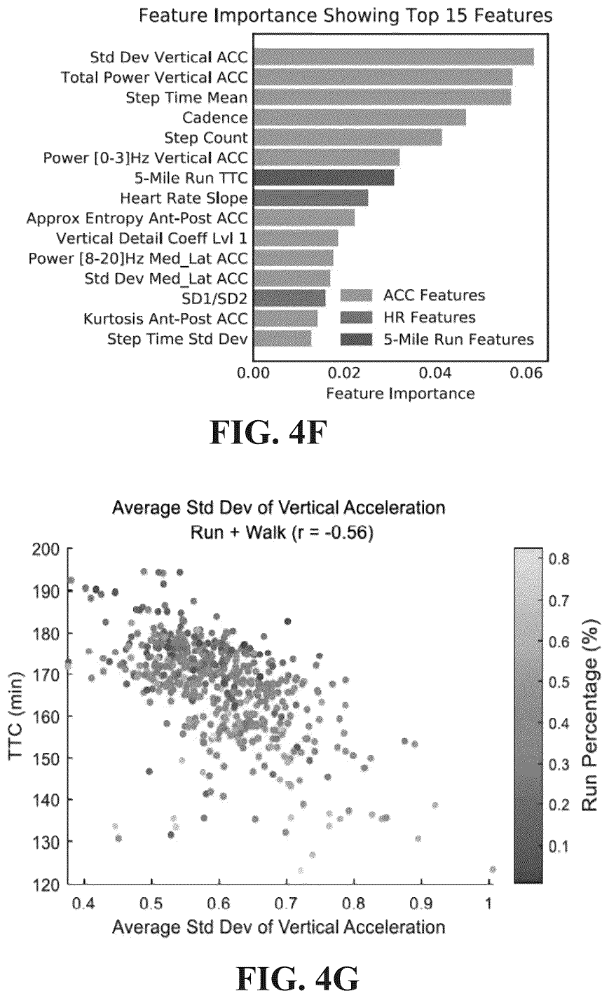

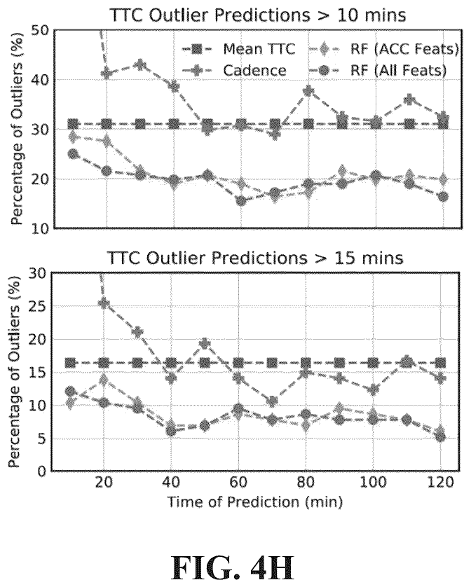

Model for experiments. FIG. 4A shows the exemplary model for TTC estimation implemented in the experiments. In FIG. 4A, the model defined a 12-mile ruck march 401 for each subject (e.g., athlete, soldier). The model was divided into equal segments of length At (e.g., 402a-402e), called checkpoints CP (shown as CP₁, CP₂, . . . , CP_{N}). Signals within each checkpoint CPᵢ were then used to calculate features 404 and subsequently combined with 5-mile run-derived features 406 to form the feature vector 408 (denoted as Fᵢ or F₁, F₂, . . . , F_{N}) for CPᵢ. The associated model at CPᵢ (e.g., 410a-410d) was then trained on the feature set [Fᵢ, Fᵢ₋₁, F₁] corresponding to features from the current, previous, and baseline checkpoints to estimate the TTC at that point in time (e.g., 412a-412d). At was set to 10 minutes.

The 12-mile ruck march duration 401 is first divided into standard epochs. The epochs are referred to as “checkpoints” (CP), which serve as reference gates for predicting TTC. For each subject, features Fᵢ for each checkpoint CPᵢ was formed using both the signals 404 measured at that checkpoint and from the features 406 associated with the 5-mile run. These “global” 5-mile run features 406 provide previous knowledge of soldier fitness. Subjects who did not have a 5-mile run were not included in the current analysis.

In the exemplary model shown in FIG. 4A, a 10-minute long epoch was chosen as the checkpoint length to provide TTC estimates at approximately every 5% of the total expected march duration. A parametric study of checkpoint length was conducted, and a 10-minute checkpoint length offer a reasonable compromise between computational cost and prediction accuracy, with shorter checkpoints performing similarly and longer checkpoints reducing prediction accuracy. TTC 412a-412d was then estimated at each checkpoint by inputting features 408 from multiple checkpoints into a distinct random forest regression model [19]. Specifically, the features from the current checkpoint (Fᵢ), previous checkpoint (Fᵢ₋₁), and baseline checkpoint (F₁) were concatenated together and used as input to the corresponding model. Here, the baseline feature matrix was the feature matrix before the first checkpoint. The model included the features, an estimated time-to-completion (TTC) determined at a given segment of the activity (e.g., time duration) as the user in moving a distance from a start position to a position in the given segment through the plurality of geographic checkpoints as defined by the segments, wherein each respective trained AI model of the one or more trained ML models was trained on one or more physiological signals for a set of users (preferably a different set of users than the current user) moving through a set of geographic checkpoints up and including to the segment.

Random forest regression was chosen for its ability to explain non-linear relationships in an explainable model using feature importance [20]. For the exemplary model shown in FIG. 4A, the study used the Gini importance index to estimate the relative importance of each feature to TTC estimation performance [19].

Feature Extraction. FIG. 4B shows the feature extraction pipeline (shown as 404 in FIG. 4A) of the exemplary model, providing a detailed explanation of how features were extracted from the 12-mile ruck march and 5-mile run data.

For the 12-mile march, each 10-minute checkpoint was partitioned into non-overlapping windows of 30 seconds, 10 seconds, and 15 seconds for heart rate (HR) 422, acceleration (ACC) 420, and skin temperature (SKT) 424, respectively. The length of HR window 422 was longer in order to have sufficient samples due to the lower sampling rate. For ACC specifically, 10 s windows 420 were used to provide computationally efficient but localized estimates of gait parameters and meet the minimum prescribed window size of approximately 5 s from [21] for accurately estimating step count. A 15-second window 424 for skin temperature measurements was selected to account for the lower sampling rate of SKT. The 12-mile feature matrix 428 contained a total of 21 HR features, 58 ACC features, and 1 temperature feature tracked over all available windows. A single checkpoint feature was calculated as the average (430) of features across all windows contained within the checkpoint.

The features from the acceleration signals included statistical measures (mean, standard deviation, and kurtosis) and frequency band powers for each of the three axes. The approximate entropy and multiscale wavelet coefficients [22], as well as frequency power features, were also collected. Notably, the vertical acceleration axis was used to calculate gait-related parameters such as cadence, step count, step time, coefficient of variation, step regularity, stride regularity, step symmetry, and step asymmetry [23]. Features for the heart rate signal included heart rate mean and frequency power band features. In addition, heart rate variability (HRV) metrics, such as the Poincare parameters, were also computed [15]. Heart rate slope was also calculated as the change in average heart rate between successive windows over the window length to provide a measure of heart rate dynamics. Finally, the core and skin temperature difference was calculated as a metric of heat strain compensation [24].

To incorporate prior knowledge of soldier fitness, features 406 from each soldier's corresponding 5-mile run were also taken into consideration. Specifically, the TTC for the 5-mile run (TTC-5m) and the time constants from the post-exercise heart rate recovery models 406 [25-26] were added to the 12-mile feature matrix 428 in a concatenation operation 432 as global features. This global TTC-5m 406 was chosen as a baseline of a subject's general performance, while the heart rate recovery constants were included after the work of Pierpont et al., which demonstrated that they may be used as an index of sympathetic withdrawal and parasympathetic reactivation after strenuous activity [25]. These features 406 were appended onto the 12-mile march feature matrix 428 for each subject to generate a feature vector 434 (denoted as Fᵢ, shown as 408 in FIG. 4A).

TTC Label Annotation. Since GPS information was not available for the data used in this study, TTC was manually calculated for each soldier using the raw vertical ACC and HR signals for both the 5-mile run and 12-mile march. Time boundaries were visually extracted using changes in energy between minimal activity (e.g., standing) and exertion at the start and end of both the march and run. The TTC for a given activity was then computed as the difference between these boundaries. The final TTC for a participant was then calculated by taking the average of the acceleration and heart rate-derived TTCs. Soldiers with a TTC of less than 120 minutes were removed from the analysis. This threshold was set because a TTC of less than 120 minutes can be considered to be physically unrealistic for the 12-mile march. Indeed, the subjects that were removed based on this threshold were either those whose sensor data were corrupt or who were extracted from the field for medical reasons (e.g., 1 subject experienced heat stroke). This resulted in the final subject count of (N=468).

Machine learning model training. Random forest training and testing were done using a 75/25 train-test split, randomized by subject. Training and testing splits were consistent across each checkpoint model to ensure the same subjects were always in the same testing or training sets. In addition, each model was only trained on subjects who had not finished at that checkpoint. The model at each checkpoint had an ensemble of 200 trees, trained with bagging. An inner loop 3-fold cross-validation was used to fit hyperparameters such as maximum depth ranging from 10 to 100 and maximum number of features to split on [19].

The study investigated the effect of two parameters integral to the exemplary model architecture: (1) the addition of historic feature information and (2) the use of a distinct model for each checkpoint.

The additional feature matrices from the previous and baseline checkpoints were included to provide a history of the extracted features. These additional features may provide context to the current features, such as a period of recovery after high strain. These learned contexts may improve TTC estimation.

A unique random forest model was used for each checkpoint to leverage the structured nature of the exercise and provide context to acquired features. This may be used to identify whether regions of high strain are due to the terrain being physically draining or due to the increased performance of subjects with low TTC. In addition, important features that predict TTC may evolve throughout the exercise, necessitating the need to reevaluate the exemplary model.

Prediction error. For all tests conducted, the study analyzed performance using 4 different TTC prediction methods: (1) estimates using the mean of the TTC labels, (2) estimates using a model based on cadence and stride length model, (3) estimates using the exemplary RF regression model with only acceleration features provided, and (4) estimates using the exemplary RF regression model with all features provided. The study referred to the method using the mean TTC for TTC estimation as the Mean TTC method and the method using cadence as the Cadence method.

The cadence-based model estimates TTC at each checkpoint using Equation 1 with cadence and stride length as a velocity surrogate (Vᵢ).

$${TTC}_{i} = \frac{{D}_{tot} - Δ t {\sum }_{k}^{i} ( {V}_{k} )}{{V}_{i}} + i Δ t \qquad ( Eq . 1 )$$

The distance already traveled by a soldier was calculated by multiplying the velocities at all prior checkpoints and the duration of each checkpoint (Δt). These two components were used to estimate the remaining time to complete the march. The elapsed time (iΔt) was then added to output the final TTC. The total march distance (Dₜₒₜ) was 12 miles in this study.

Cadence was calculated at each checkpoint; however, because stride length estimation was not well-researched without gyroscope measurements [11], [13], a parameter study was conducted to find the optimal stride. Specifically, stride lengths from 75-90 cm were used to estimate TTC on the training set. In this study, a stride length of 86 cm resulted in the lowest RMSE, which was similar to the average stride length of an average military soldier [27]. This was then used as the stride length on the test set.

The comparison of the first 2 models was conducted to investigate whether any significant prediction changes occur from the addition of physiological features. The cadence-based model was used to compare the exemplary method with a simple velocity-based TTC estimation model. The study only showed TTC estimations up to the 120-minute mark (i.e., 66% of the permitted march time) to highlight model performance early into the march. This time limit matched the lowest TTC in the data (fastest subject) and ensured that all TTC models contained the same number of test subjects. Specifically, at checkpoints past this time limit, the TTC labels being estimated naturally had a larger mean and lower standard deviation as soldiers finish the march. As such, comparisons were limited to before this time limit to ensure all models were evaluated using the same TTC label distribution.

FIG. 4C shows the root mean square error (RMSE) and absolute error for the time-to-completion (TTC) prediction of 4 models, e.g., mean TTC, cadence-based, exemplary method with only acceleration features (shown as RF (ACC features), and exemplary method with all the features (shown as RF (all features).

Subpanel (a) shows the root mean square error (RMSE) between the predicted TTC estimates and ground truth TTC labels for all tested models. While the RMSE error using the Mean TTC-based method remained at ≈11.70 minutes, the RMSE when using the RF model was lower, steadily decreasing, at 120 minutes to 7.07 minutes when using all features and 7.73 when using just acceleration features. The addition of the physiological features reduced Mean TTC RMSE with a maximum difference of 0.8 minutes, suggesting some benefit of including these modalities for this task. The cadence-based model also showed better performance than when using the Mean TTC at later checkpoints. However, the exemplary model still performed better, suggesting that there was additional information in the acceleration and physiological signals for better estimating TTC. The cadence-based model had an RMSE of 27.83 minutes at CP, (shown in subpanel a), a value much greater than the RMSE at other checkpoints. The high RMSE was determined to be due to a larger estimated cadence compared to the other checkpoints. This coupled with the assumption that the estimated velocity remain constant for the remainder of the march, caused TTC1 calculations to heavily underestimate the true TTC.

Subpanel (b) shows the box plots for the absolute TTC error with absolute error means and medians listed in Table 1 for a few prediction times. Table 1 shows the median (mean) error of |True TTC−Estimated TTC| using 4 models, e.g., Mean TTC, cadence-based, exemplary method with acceleration (ACC) feature only, and exemplary method using all features.

TABLE 1

| | | TTC | | |
|---|---|---|---|---|
| | TTC | using | TTC | TTC |
| | using | Cadence- | using | using |
| Time of | Mean | based | ACC | all |
| Prediction | TTC | estimate | features | features |
| [mins] | [mins] | [mins] | [mins] | [mins] |
| 10 | 7.45 (8.95) | 26.57 (25.21) | 6.25 (7.35) | 5.50 (7.19) |
| 40 | 7.45 (8.95) | 7.57 (8.76) | 4.98 (6.18) | 5.13 (6.15) |
| 80 | 7.45 (8.95) | 7.08 (8.33) | 3.85 (5.86) | 4.03 (5.78) |
| 120 | 7.45 (8.95) | 7.01 (8.13) | 4.41 (5.74) | 4.03 (5.28) |

As shown in Table 1, as the exercise progressed, the mean and median absolute error of both the exemplary RF regressors decreased. From the box plots shown in subpanel (b), the upper whisker and the upper quartile for the RF with all features included were lower than its RF with only acceleration features counterpart. However, this relationship was not significant.

FIG. 4D shows the modified Bland-Altman and correlation plots for estimated TTC using both the exemplary RF configurations. TTC estimates at 120 minutes were included. As shown in FIG. 4D, errors were symmetrical with a slight negative skewness at lower TTC values. Subjects with the fastest (<130 min) and slowest (>180 min) completion times had the highest prediction error, potentially attributed to the relatively small number of training examples that completed the march in these times. Although the 95% limits of agreement may seem high, it corresponded to an error of <10% for a total expected march time of 180 minutes. Furthermore, the high correlation between the estimated and true TTC suggested that although TTC cannot be precisely estimated, the exemplary model can still stratify low and high-performing subjects, which was also important for leadership feedback on training effectiveness.

Effect of model data inputs. To motivate the increased complexity of (1) including features from previous checkpoints and (2) training an individual RF regression model for each checkpoint, additional model configurations were trained to investigate the effects of each change individually. These models were then compared with the exemplary configuration of using separate RF models for each checkpoint and a feature construction using [CP₁, CPₙ₋₁, CPₙ] referring to features derived from the baseline, previous, and current checkpoint, respectively. Models were evaluated using TIC RMSE at each checkpoint.

To test the advantages of including features from previous checkpoints for TTC estimation, the exemplary RF models were trained at each checkpoint using different feature construction setups. Specifically, models were trained at each checkpoint with features from just the current checkpoint [CPₙ], features from the current and previous checkpoint [CPₙ₋₁, CPₙ], and features from the current and baseline checkpoints [CP₁, CPₙ]. The study referred to these tests as Multi RF-Multi CP X where X was the feature construction configuration.

To test the benefit of training an individual model for each checkpoint, a single model (Single RF-Multi CP) was trained to predict over the entire march period. This model was trained using a feature construction of [CP₁, CPₙ₋₁, CPₙ] from every checkpoint. Predictions were not made at 10 or 20 minutes for this model because not enough checkpoints were available to feed into the model.

FIG. 4E shows the TTC RMSE while varying the input feature matrix or the number of models trained to predict TTC. Using the Multi RF-Single CP [CPₙ] model configuration reduced performance. However, with the inclusion of the features from the baseline checkpoint, Multi RF-Multi CP [CP₁, CPₙ] showed improved performance at each checkpoint. TTC RMSE was further improved when including just the previous checkpoint with Multi RF-Multi CP [CPₙ₋₁, CPₙ], showing the best results compared to the previous two models tested. This suggested that better TTC estimation at a point in the march required not only current physiological and movement data but also past data.

Using the Single RF-Multi CP configuration maintained consistent performance throughout the march at a higher RMSE than the exemplary method. The increase in RMSE suggested that different environmental factors throughout the march may have an effect on TTC estimation. For example, high strain periods in physiological measurements may be due to general fatigue that affected lower-performing soldiers or large changes in inclines that affected all participating soldiers. As such, dividing the march into distinct checkpoints implicitly provided environmental context that was common between all soldiers tested.

Feature importance. FIG. 4F shows the top 15 features across all trained models. The most important feature was the average standard deviation of vertical acceleration and the average power of vertical acceleration. Both features indicated a sense of force applied in the vertical direction. Additionally, many of the top features were gait-related, such as step time, cadence, and step count, which were important parameters for calculating distance and speed.

FIG. 4G shows further investigation into the relationship between high vertical acceleration standard deviation and TTC, color-coded by the percentage of windows that the subject spent running. In this case, the running and walking boundary was defined on a subject-by-subject case using cadence as a discriminatory feature [28]. As shown in FIG. 4G, there was a negative correlation between TTC and vertical acceleration standard deviation. In addition, subjects with a high variance of vertical acceleration were usually associated with a high percentage of running windows. This indicated that increased vertical acceleration was closely related to high cadence, both of which contributed to a lower TTC.

The study also observed that the 5-mile TTC was useful in predicting the 12-mile TTC, suggesting that prior exercise information can be useful for predicting TTC in another exercise.

The heart rate slope was useful in predicting TTC. HR and HRV features have been shown to be indicators of energy expenditure [29]. However, there were fewer high-contributing HR features compared to ACC features, indicating that acceleration features were more important for predicting TTC.

Further of interest was that although Estimated Core Temperature (ECT) and Skin Temperature (SKT) were included as features for TTC estimation based on the fact that they served as important markers for fitness in the context of heat exertion, these features did not affect TTC prediction, and did not factor in the top 15 contributing features of exemplary models.

Outlier analysis. To analyze model performance, the study examined the number of TTC predictions that were skewed from the true TTC values. FIG. 4H shows the percentage of subjects at each checkpoint whose absolute TTC prediction error exceeded a threshold of 10 or 15 minutes. Ten minutes was chosen as a threshold to match the march segmentation duration, while 15 minutes was chosen to contain 1 standard deviation of the overall TTC distribution (166.86±11.89 mins). Both models had fewer outliers compared to using the Mean TTC method. There was also a downward trend of outliers as the exercise progressed, indicating the model became more accurate over time. While the outlier percentage using a threshold of 10 minutes dropped from 25% to 15% with the exemplary model, the outlier percentage using 15 minutes dropped from 11% to 5%. This discrepancy of outliers between the two thresholds suggested that a large portion of outliers were within the error range of 10 to 15 minutes. These outliers corresponded to subjects with a TTC in the higher or lower range, as shown in FIG. 4D. The exemplary model estimated TTC poorer on these subjects than those with moderate TTC because of the low number of available subjects in the TTC upper and lower ranges. More data with TTC labels at the extremities may be needed to enable the model to learn behaviors at these ranges.

### DISCUSSION

Discussion #1. Assessing soldier fitness is important to identify unit readiness during training and prior to deployment. Further, individual soldier fitness assessments can inform military leadership of training regimen optimizations over personal to regiment levels. The United States (US) Army conducts regular fitness tests [1] for soldiers that comprise a series of exercises such as general physical training, timed runs, and timed ruck marches. These are currently monitored by manually keeping track of soldier performance through metrics such as the number of repetitions completed or the time taken to complete an exercise.

Ruck marches, specifically, have been shown to be effective indicators of soldier fitness as they are conducted over challenging terrain with the soldiers carrying heavy loads in excess of 25 kg on their backs, reproducing realistic operational conditions [2]. These marches are usually timed, and soldiers are required to complete a fixed distance over a specified route within the allowed time. This study refers to such marches as “structured marches.” A heuristic metric of march performance is the time taken by soldiers to complete the event or Time-to-Completion (TTC). At present, the Army measures completion time as the soldier reaches the end of the prescribed course. With access to data from portable wearables tracking accelerometry and physiology [3], near real-time updates on soldier progress through the march become possible and offer a more comprehensive picture of soldier performance. Specifically, the expected TTC for a soldier at any given march instance could be provided to the individual and/or the unit commander, and based on that value, the pace during the structured march may be increased or decreased accordingly, in real-time, to maximize the probability of finishing within the goal for TTC for that individual.

The use of a Global Positioning System (GPS) may make estimating the TTC more accurate as it can provide better estimates of velocity than direct integration of accelerometry data can. GPS sensors can be installed in wearable devices and fitted onto each soldier, enabling constant updates of TTC. However, because soldiers need to complete multiple exercises during the whole day, an unobtrusive, rugged, portable sensor with a long battery life is preferable [4], [5]; these design constraints can limit the integration of GPS into the sensing hardware from a packaging and power consumption standpoint. [6]. GPS accuracy can also degrade for various environmental reasons, including low signal-to-noise ratio (SNR), multipath errors, and limited line of sight to satellites when marching through regions of dense tree cover [7]. The accuracy can also be affected by operational errors, including improperly wearing the sensor, device misuse, or device initialization errors [8]. These limitations of GPS make it difficult to rely solely on satellite positioning for TTC prediction. As such, the ability to estimate TTC from current sensor systems that measure accelerometry and other physiological signals locally is still desired.

The exemplary system and method may use to estimate the TTC of a soldier for a structured march. Related works on estimating a completion time include stride length estimation using accelerometer data by Xing et al. [9], where a neural network trained on accelerometer features and participant height was used to estimate the distance covered per step. However, this study focused on a pedestrian population with no load carriage and required wearing the monitoring sensor on the foot. Further, in the study, soldier heights were not readily available, making direct stride length estimation using Xing et al.'s [9] method infeasible. Moreover, information from physiological measurements was not used, and stride estimation was achieved using gyroscopes. While gyroscopes are generally useful for activity monitoring and stride length estimation [10], [11], this sensing modality was not available during this study.

There are, however, alternative measures that may be used to predict performance for a soldier over a structured march using only accelerometer and physiological data. For example, heart rate can be a proportional measure of metabolic energy expenditure [12], at a given point during the march, while skin and core temperature measurements can indicate physical exertion [14]. Moreover, the maximization of lateral acceleration and the minimization of vertical acceleration have been shown to improve gait efficiency [15], suggesting the use of acceleration power as an efficient indicator of performance. Studies have also shown that high-performing athletes are able to maintain lower heart rates for long durations of time during endurance training [16], [17], suggesting that heart rate dynamics could be used as a metric of march performance. A predictive model to estimate TTC can be developed using a collection of such derived features, where the model can infer relative feature importance based on the sensor data available when making its prediction. This semi-empirical approach directly handles the complex inter-relationship between gait parameters and physiology per individual over the march.

In contrast, the exemplary model uses physiological measurement (skin temperature, heart rate, and estimated core temperature) and triaxial accelerometry from wearable sensors to accurately predict TTC over a structured march. The total length of the course is divided into checkpoints, and at each checkpoint, a new prediction of the TTC is made. A random forest regressor at each checkpoint is trained to provide this new TTC prediction using features not only from the current checkpoint but also from previous checkpoints in the march. The model is trained and tested on a large population of soldiers performing a structured march.

To summarize, the exemplary method employs a physiology-driven Time-to-Completion prediction model that serves as a metric for structured march performance.

Discussion #2. The study demonstrated a TTC prediction framework for structured marches using simple measurements of acceleration, heart rate, and skin temperature from portable wearable devices. The study achieved an average absolute error of 5.23 minutes by ⅔ of the expected completion time with few outlier predictions. The use of separate models for each checkpoint takes advantage of the march's structured nature to reduce the prediction error as the march progresses. The exemplary model can be trained on an incoming group of soldiers and be used for longitudinal tracking of TTC as they go through these structured marches multiple times during basic training. Such performance metrics are important for military personnel to evaluate the physical capabilities and readiness of soldiers before field deployment.

While discussed in relation to military ruck marches, the exemplary method and corresponding system can be extended to other structured athletics disciplines, such as cross-country and track-and-field. For these situations and when applying the proposed methodology to other disciplines, new training datasets representative of the exercise being performed would be necessary to achieve the desired results.

### CONCLUSION

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products, including machine-readable media for carrying or having machine-executable instructions or data structures stored thereon. Such machine-readable media can be any available media that can be accessed by a general-purpose or special-purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium, thus, any such connection is properly termed a machine-readable medium. Combinations of the above are also included within the scope of machine-readable media. Machine-executable instructions include, for example, instructions and data that cause a general-purpose computer, special-purpose computer, or special-purpose processing machine to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on the designer's choice. All such variations are within the scope of the disclosure. Likewise, software implementations could be accomplished with programming techniques with rule-based logic and other logic to accomplish the various connection steps, processing steps, comparison steps, and decision steps.

Machine Learning. In addition to the machine learning features described above, the analysis system can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

The following patents, applications and publications as listed below and throughout this document are hereby incorporated by reference in their entirety herein.
[1] J. J. Knapik, W. Rieger, F. Palkoska, S. V. Camp, and S. Darakjy, “United States Army Physical Readiness Training: Rationale and Evaluation of the Physical Training Doctrine,” J. Strength Cond. Res., vol. 23, no. 4, pp. 1353-1362 July 2009, doi: 10.1519/JSC.0b013e318194df72.
[2] T. Wyss, J. Scheffler, and U. Mader, “Ambulatory Physical Activity in Swiss Army Recruits,” Int. J. Sports Med., vol. 33, no. 9, pp. 716-722, September 2012, doi: 10.1055/s-0031-1295445.
[3] M. Cardinale and M. C. Varley, “Wearable Training-Monitoring Technology: Applications, Challenges, and Opportunities,” Int. J. Sports Physiol. Perform., vol. 12, no. s2, pp. S2-55-S2-62, April 2017, doi: 10.1123/ijspp.2016-0423.
[4] W. J. Tharion and R. W. Hoyt, “Form Factor Evaluation of Open Body Area Network (OBAN) Physiological Status Monitoring (PSM) System Prototype Designs,” U.S. Army Research Institute of Environmental Medicine Natick United States, May 2018. Accessed: Jun. 29, 2022.
[5] A. Ometov et al., “A Survey on Wearable Technology: History, State-of-the-Art and Current Challenges,” Comput. Netw., vol. 193, p. 108074, July 2021, doi: 10.1016/j.comnet.2021.108074.
[6] S. Seneviratne et al., “A Survey of Wearable Devices and Challenges,” IEEE Commun. Surv. Tutor., vol. 19, no. 4, pp. 2573-2620, 2017, doi: 10.1109/COMST.2017.2731979.
[7] E. Abdi, H. S. Mariv, A. Deljouei, and H. Sohrabi, “Accuracy and precision of consumer-grade GPS positioning in an urban green space environment,” For. Sci. Technol., vol. 10, no. 3, pp. 141-147, July 2014, doi: 10.1080/21580103.2014.887041.
[8] V. A. Paz-Soldan et al., “Strengths and Weaknesses of Global Positioning System (GPS) Data-Loggers and Semi-structured Interviews for Capturing Fine-scale Human Mobility: Findings from Iquitos, Peru,” PLOS Negl. Trop. Dis., vol. 8, no. 6, p. e2888, June 2014, doi: 10.1371/journal.pntd.0002888.
[9] H. Xing, J. Li, B. Hou, Y. Zhang, and M. Guo, “Pedestrian Stride Length Estimation from IMU Measurements and ANN Based Algorithm,” J. Sens., vol. 2017, p. e6091261, February 2017, doi: 10.1155/2017/6091261.
[10] A. Köse, A. Cereatti, and U. Della Croce, “Bilateral step length estimation using a single inertial measurement unit attached to the pelvis,” J. NeuroEngineering Rehabil., vol. 9, no. 1, p. 9, February 2012, doi: 10.1186/1743-0003-9-9.
[11] Y. Mao, T. Ogata, H. Ora, N. Tanaka, and Y. Miyake, “Estimation of stride-by-stride spatial gait parameters using inertial measurement unit attached to the shank with inverted pendulum model,” Sci. Rep., vol. 11, no. 1, Art. no. 1, January 2021, doi: 10.1038/s41598-021-81009-w.
[12] S. E. Crouter, J. R. Churilla, and D. R. Bassett, “Estimating energy expenditure using accelerometers.” Eur J Appl Physiol 98, 601-612 (2006). doi: 10.1007/s00421-006-0307-5.
[13] H. Xing et al., “Pedestrian Stride Length Estimation from IMU Measurements and ANN Based Algorithm.”
[14] M. J. Buller et al., “Estimation of human core temperature from sequential heart rate observations,” Physiol. Meas., vol. 34, no. 7, p. 781, June 2013, doi: 10.1088/0967-3334/34/7/781.
[15] C. A. Clermont, L. C. Benson, W. B. Edwards, B. A. Hettinga, and R. Ferber, “New Considerations for Wearable Technology Data: Changes in Running Biomechanics During a Marathon,” J. Appl. Biomech., vol. 35, no. 6, pp. 401-409, December 2019, doi: 10.1123/jab.2018-0453.
[16] D. Herzig, B. Asatryan, N. Brugger, P. Eser, and M. Wilhelm, “The Association Between Endurance Training and Heart Rate Variability: The Confounding Role of Heart Rate,” Front. Physiol., vol. 9, 2018, Accessed: May 27, 2022.
[17] A. L. Baggish and M. J. Wood, “Athlete's Heart and Cardiovascular Care of the Athlete,” Circulation, vol. 123, no. 23, pp. 2723-2735 June 2011, doi: 10.1161/CIRCULATIONAHA.110.981571.
[18] B. A. Telfer, K. Byrd, and P. P. Collins, “Open Body Area Network Physiological Status Monitor,” vol. 24, no. 1, p. 17, 2020.
[19] L. Breiman, “Random Forests,” Mach. Learn., vol. 45, no. 1, pp. 5-32, October 2001, doi: 10.1023/A: 1010933404324.
[20] U. Gromping, “Variable Importance Assessment in Regression: Linear Regression versus Random Forest,” Am. Stat., vol. 63, no. 4, pp. 308-319, November 2009, doi: 10.1198/tast.2009.08199.
[21] E. Fortune, V. Lugade, M. Morrow, and K. Kaufman, “Validity of Using Tri-Axial Accelerometers to Measure Human Movement-Part II: Step Counts at a Wide Range of Gait Velocities,” Med. Eng. Phys., vol. 36, no. 6, pp. 659-669, June 2014, doi: 10.1016/j.medengphy.2014.02.006.
[22] M. Costa, C.-K. Peng, A. L. Goldberger, and J. M. Hausdorff, “Multiscale entropy analysis of human gait dynamics,” Phys. Stat. Mech. Its Appl., vol. 330, no. 1, pp. 53-60, December 2003, doi: 10.1016/j.physa.2003.08.022.
[23] R. Moe-Nilssen and J. L. Helbostad, “Estimation of gait cycle characteristics by trunk accelerometry,” J. Biomech., vol. 37, no. 1, pp. 121-126, January 2004, doi: 10.1016/S0021-9290(03)00233-1.
[24] M. N. Sawka and A. J. Young, “Physiological Systems and Their Responses to Conditions of Heat and Cold,” Army Research Inst of Environmental Medicine Natick MA Thermal and Mountain Medicine Division, January 2006. Accessed: Jun. 12, 2022.
[25] G. L. Pierpont, D. R. Stolpman, and C. C. Gornick, “Heart rate recovery post-exercise as an index of parasympathetic activity,” J. Auton. Nerv. Syst., vol. 80, no. 3, pp. 169-174, May 2000, doi: 10.1016/S0165-1838(00)00090-4.
[26] G. L. Pierpont and E. J. Voth, “Assessing autonomic function by analysis of heart rate recovery from exercise in healthy subjects,” Am. J. Cardiol., vol. 94, no. 1, Art. no. 1, July 2004, doi: 10.1016/j.amjcard.2004.03.032.
[27] P. E. Martin and R. C. Nelson, “The effect of carried loads on the walking patterns of men and women,” Ergonomics, vol. 29, no. 10, pp. 1191-122 October 1986, doi: 10.1080/00140138608967234.
[28] C. Chase, “Cadence as an Indicator of the Walk-to-Run Transition”, doi: 10.7275/16698528.
[29] F. Shaffer and J. P. Ginsberg, “An Overview of Heart Rate Variability Metrics and Norms,” Front. Public Health, vol. 5, 2017, Accessed: May 27, 2022.
[30] C. Rich et al., “Quality Control Methods in Accelerometer Data Processing: Identifying Extreme Counts” PloS one 9.1 (2014): e85134.

What is claimed is:

1. A method comprising:
receiving, by a processor, signals from a wearable sensor device worn by a user during an activity, wherein the activity is defined by the user moving through a plurality of geographic checkpoints at a first location, including a first checkpoint and a second checkpoint; and
determining, via one or more trained AI models or a model derived therefrom, an estimated time-to-completion (TTC) determined at a given segment of the activity as the user in moving a distance from a start position to a position in the given segment through the plurality of geographic checkpoints as defined by the segments, wherein each respective trained AI model of the one or more trained AI models was trained on one or more physiological signals for a set of users moving through a set of geographic checkpoints up and including to the segment,
wherein the estimated time-to-completion of the activity or an estimated complete time derived therefrom is outputted to be displayed at the wearable sensor device or an external remote device.

2. The method of claim 1, wherein the estimated time-to-completion of the activity or the estimated complete time derived therefrom is outputted to a cloud network, wherein the cloud network transmits the estimated time-to-completion of the activity or the estimated complete time to the wearable sensor device or the remote device for display.

3. The method of claim 1, wherein the determining the estimated TTC for the given segment of the activity is performed at the wearable sensor device.

4. The method of claim 1, wherein the one or more physiological signals are selected from the group consisting of a heart rate signal, a temperature signal, an accelerometer signal, a body signal.

5. The method of claim 1, wherein the one or more trained AI models include a machine learning model or a neural network model.

6. The method of claim 1, wherein the estimated time-to-completion of the activity is defined as an average predicted duration of remaining time to complete the activity as of that given segment, wherein the checkpoint or segment is determined only by the signals from the wearable sensor device.

7. The method of claim 1, wherein the determining the estimated TTC of ethe given segment of the activity is performed at a computing device located in cloud infrastructure.

8. The method of claim 1, wherein the determining the estimated TTC of the given segment of the activity is performed at a remote computing device.

9. An analysis system comprising:
a processor; and
a memory having instructions stored thereon, wherein execution of the instructions by the processor causes the processor to:
receive, by a processor, signals from a wearable sensor device worn by a user during an activity, wherein the activity is defined by the user moving through a plurality of geographic checkpoints at a first location, including a first checkpoint and a second checkpoint; and
determine, via one or more trained AI models or a model derived therefrom, an estimated TTC determined at a given segment of the activity as the user in moving a distance from a start position to a position in the given segment through the plurality of geographic checkpoints as defined by the segments, wherein each respective trained AI model of the one or more trained AI models was trained on one or more physiological signals for a set of users moving through a set of geographic checkpoints up and including to the segment, wherein the one or more trained ML models are trained on one or more physiological signals for a set of users moving through a set of geographic checkpoints corresponding to the segment,
wherein the estimated time-to-completion of the activity or an estimated complete time derived therefrom is outputted to be displayed at the wearable sensor device or an external remote device.

10. The analysis system of claim 9, wherein the estimated time-to-completion of the activity or an estimated complete time derived therefrom is outputted to be displayed on a network interface.

11. The analysis system of claim 9, wherein the wearable sensor device comprises:
one or more sensors configured to measure the one or more physiological signals for a set of users moving through a set of geographic checkpoints at a plurality of locations.

12. The analysis system of claim 11, wherein the one or more physiological signals are selected from the group consisting of a heart rate signal, a temperature signal, an accelerometer signal, a body signal, or a combination thereof.

13. The analysis system of claim 9, wherein the determining the estimated TTC of the given segment of the activity is performed at a computing device located in cloud infrastructure.

14. The analysis system of claim 9, wherein the determining the estimated TTC of the given segment of the activity is performed at a remote computing device.

15. A non-transitory computer-readable medium having instructions stored thereon, wherein execution of the instructions by a processor causes the processor to:
receive, by a processor, signals from a wearable sensor device worn by a user during an activity, wherein the activity is defined by the user moving through a plurality of geographic checkpoints at a first location, including a first checkpoint and a second checkpoint; and
determine, via one or more trained AI models or a model derived therefrom, an estimated TTC determined at a given segment of the activity as the user in moving a distance from a start position to a position in the given segment through the plurality of geographic checkpoints as defined by the segments, wherein each respective trained AI model of the one or more trained ML models was trained on one or more physiological signals for a set of users moving through a set of geographic checkpoints up and including to the segment,
wherein the estimated time-to-completion of the activity or an estimated complete time derived therefrom is outputted to be displayed at the wearable sensor device or an external remote device.

16. The non-transitory computer-readable medium of claim 15, wherein the estimated time-to-completion of the activity or an estimated complete time derived therefrom is outputted to be displayed on a network interface.

17. The non-transitory computer-readable medium of claim 15, wherein the wearable sensor device comprises:
one or more sensors configured to measure the one or more physiological signals for a set of users moving through a set of geographic checkpoints at a plurality of locations.

18. The non-transitory computer-readable medium of claim 17, wherein the one or more physiological signals are selected from the group consisting of a heart rate signal, a temperature signal, an accelerometer signal, a body signal or a combination thereof.

19. The non-transitory computer-readable medium of claim 15, wherein the determining the estimated TTC of the given segment of the activity is performed at a computing device located in cloud infrastructure.

20. The non-transitory computer-readable medium of claim 15, wherein the determining the estimated TTC of the given segment of the activity is performed at a remote computing device.
